(19) United States
(12) Patent Application Publication
(10) Pub. No.: US 2026/0083451 A1
(43) Pub. Date: Mar. 26, 2026

(54) MODULAR DEFORMABLE SUTURE NEEDLE FOR MINIMALLY INVASIVE SURGERY AND FORM CONTROL METHOD THEREOF

(71) Applicant: Jiangsu Cancer Hospital, Nanjing City (CN)

(72) Inventors: Gang LI, Nanjing City (CN); Lingli HUANG, Nanjing City (CN); Tong LI, Nanjing City (CN)

(21) Appl. No.: 19/240,390

(22) Filed: Jun. 17, 2025

(30) Foreign Application Priority Data
Sep. 25, 2024 (CN) 202411342602.5

(51) Int. Cl. A61B 17/04; A61B 17/00; A61B 17/06; G16H 40/63

(52) U.S. Cl. CPC A61B 17/0469; A61B 17/06066; G16H 40/63; A61B 2017/00017; A61B 2017/00862; A61B 2017/00867; A61B 2017/00955

(57) ABSTRACT

The present invention relates to the field of medical instruments, in particular to a modular deformable suture needle for a minimally invasive surgery and a form control method thereof. Through connecting deformation control units with position differences in parallel, a small diameter of a needle body is achieved on the premise that the overall safety is guaranteed. Needle body segments have independent controllable continuous deformation capacities in all degrees of freedom and all directions, and can quantitatively control dynamic deformations in the body of a surgical subject through an external voltage or heat power input in the suturing process, achieving high suturing efficiency and high device deformation capacity. The main steps are as follows: constructing a flexible deformation needle body composed of parallel units with position differences; constructing a modular deformable suture needle for a minimally invasive surgery; quantitatively expressing bending deformation features; establishing a single-segment form control model; and performing coherent form control. The limitations that a traditional minimally invasive surgical instrument is difficult to smoothly turn, move forward, move backward or make other actions in a complicated environment in the body of a surgical subject are overcome.

### TECHNICAL FIELD

The present invention relates to the technical field of medical devices, in particular to a modular deformable suture needle for minimally invasive surgery and a form control method thereof.

### BACKGROUND ART

Minimally invasive surgeries mainly include laparoscopic and endoscopic surgeries, and a suturing utensil is inserted from a narrow wound to perform a suturing operation, which has the characteristic of small wound and is conducive to the postoperative recovery of patients. However, when a region to be sutured is blocked or covered by tissues and organs, it is difficult for the traditional suture needle to smoothly turn, move forward, and move backward or make other actions in such complex environment, resulting in difficulty in suturing.

A suture needle with a wavy shape (having an application number of 202030050860.2, application date of Feb. 13, 2020, and authorized announcement number of CN 305950465 S) proposes a wavy needle body with a fixed form, but the form of the needle body is hardly adapted according to actual needs in the suturing process.

A surgical spiral puncture suture needle (having an application number of 202410760769.7, application date of Jun. 13, 2024, and authorized announcement number of CN 118526241 A) proposes a spiral needle body with a fixed form, but the form of the needle body is hardly adapted according to actual needs in the suturing process.

A double-fold eyelid suture needle with various specifications (having an application number of 202121606808.6, application date of Jul. 15, 2021, and authorized announcement number of CN 214856994 S) proposes a split suture needle in a fixed form, but the form of a needle body is hardly adapted according to actual needs in the suturing process.

A bendable suture needle (having an application number of 201930457152.8, application date of Aug. 22, 2019, and authorized announcement number of CN 305576726 S) cannot be disengaged from a bending load applicator in the suturing process so as to dynamically adjust a bending form.

In order to achieve the flexible deformation of a slender arm in a complex suture environment, the commonly used deformation control methods include motor drive, hydraulic drive, pneumatic drive, rope drive, and memory alloy drive. The motor drive, the hydraulic drive and the pneumatic drive are highly accurate in control, but are difficult to implement in a tiny size. The rope drive and the memory alloy drive are poorly accurate in control, and are difficult to carry out complex deformations in multiple degrees of freedom because they are both made of homogeneous flexible materials.

Pneumatic and drive-by-wire deformation systems in a flexible mechanical arm of a pneumatic and rope hybrid drive type (having an application number of 202410723788.2, application publication number of CN 118544335 A and application date of Jun. 5, 2024) is difficult for the deformation control of a suture needle with a millimeter-level diameter due to the sizes of components and the strength of materials.

A miniature pipeline detection robot with a piezoelectric drive flexible spring and a control method of the miniature pipeline detection robot (having an application number of 202211639177.7, application publication number of CN 115854173 A and application date of Dec. 20, 2022) adopt a jointed form controller, and the system has poor axial toughness and integrity, and is prone to fracture when used for a suture needle having a millimeter-level diameter.

A rope-spring assembly deformation system in a rope drive flexible mechanical arm (having an application (patent) number of CN202111371348.8, application publication number of CN113894840A and application date of Nov. 18, 2021) is difficult for the deformation control of a suture needle with a millimeter-level diameter due to the sizes of components and the strength of materials.

A nickel-titanium memory alloy coil in an artificial muscle driven by a nickel-titanium memory alloy (having an application (patent) number of CN202410349164.9, application publication number of CN118238119A and application date of Mar. 26, 2024) can produce mutual heat conduction and electromagnetic induction interference in the case of micro size, resulting in poor deformation control accuracy and failure in use for the deformation control of a suture needle with a millimeter-level diameter.

An amphibious S-shaped robot based on multiple degree of freedom of flexible motion units (having an application (patent) number of CN201210336869.4, application publication number of CN102837307A and application date of Sep. 13, 2012) adopts a dielectric deformation driving device, and the system has poor axial toughness and integrity, and fails to be used for deformation control of a suture needle with a millimeter-level diameter.

An adjustable bending catheter controlled by a shape memory alloy (having an application (patent) number of CN202410660611.2, application publication number of CN118576868A and application date of May 27, 2024) adopts a symmetrical memory alloy wire arrangement method, which cannot be deformed in an “S” shape.

A flexible mechanical arm mechanism driven by a shape memory alloy wire and a flexible mechanical arm thereof (having an application (patent) number of CN201210433479.9, application publication number of CN102962850A and application date of Nov. 5, 2012) adopt a deformation control system in which segments are connected in series, and the system has poor axial toughness and integrity, and is prone to fracture when used for micro-sized devices.

In order to dynamically adjust a form of a needle body in the suturing process and adapt to a complex suture environment, it is necessary to develop a modular deformable suture needle for a minimally invasive surgery that can be deformed in an “S” shape and has strong axial toughness and integrity, and propose a supporting applicable deformation control method, so as to solve the suturing problem that a region to be sutured is blocked and covered by tissues and organs in the minimally invasive surgery.

### SUMMARY OF THE INVENTION

In order to solve the above problems, the present invention proposes the following technical solutions.

The present invention provides a modular deformable suture needle for a minimally invasive surgery, including a short needle, a flexible deformation needle body composed of parallel units with position differences, and a heat supply unit or a power supply unit, wherein
a flexible deformation needle body composed of parallel units with position differences includes a needle body sleeve, I deformation control units, and more than one line feeding unit, in which, I≥1, and if the serial number of the deformation control unit is i, 1≤i≤I;
the line feeding unit includes a suture line and a suture line sleeve, wherein the suture line sleeve wraps the suture line;
each deformation control unit includes a memory metal wire, a conductive metal wire, aerogel particles and a film sleeve, wherein the tail end of the memory metal wire is sequentially connected to the head end of the conductive metal wire to form a deformation control unit core body, the film sleeve is wrapped outside the deformation control unit core body, and the aerogel particles are filled between the film sleeve and the deformation control unit core body; when the modular deformable suture needle for the minimally invasive surgery includes the power supply unit, the deformation control unit further includes an electrical loop metal wire, and a tail end of the electrical loop metal wire is connected to a head end of the memory metal wire;
the needle body sleeve is wrapped outside the deformation control units and the line feeding unit, the electrical loop metal wire is located inside the needle body sleeve and outside the film sleeve, and a head end of the electrical loop metal wire extends to a tail end of the needle body sleeve;
the arrangement of the deformation control units in the needle body sleeve satisfies:

$$L , ( i ) > L , ( i + 1 )$$

$$s , ( i ) , ( i + 1 ) > 0 ;$$

$$s , ( i ) , ( i + 1 ) = L , ( i ) - L , ( i + 1 ) - L , ( i , 1 ) ;$$

wherein L,(i) represents a length of the i^{th} deformation control unit extending into a minimally invasive surgical wound, having a value equal to a sum of a length of the memory metal wire of the i^{th} deformation control unit extending into the minimally invasive surgical wound and a length of the conductive metal wire of the i^{th} deformation control unit extending into the minimally invasive surgical wound;
L,(i,1) represents the length of the memory metal wire of the i^{th} deformation control unit extending into the minimally invasive surgical wound;
s,(i),(i+1) represents a distance between a tail end of the memory metal wire in the i^{th} deformation control unit and a head end of the memory metal wire in the (i+1)^{th} deformation control unit;
a head end of the needle body sleeve is connected to an outer edge of a head end of the suture line sleeve in a fixed sealing manner, so that parts of the deformation control units extending into the wound are isolated from organ tissues in the body of a surgical subject by the needle body sleeve, and a line feeding unit extending into the wound is also isolated from organ tissues in the body of a surgical subject by the needle body sleeve;
a tail end of the short needle is connected to the suture line;
the heat supply unit is connected to the tail end of the conductive metal wire and used to provide a heat source;
the power supply unit is connected to the tail end of the conductive metal wire and the head end of the electrical loop metal wire and used to provide a power source;
the tail end of the memory metal wire is electrically connected to the head end of the conductive metal wire to form the deformation control unit core body of the single deformation control unit, a head end of the deformation control unit core body is the head end of the memory metal wire, and a tail end of the deformation control unit core body is the tail end of the conductive metal wire;
the tail end of the electrical loop metal wire is electrically connected to the head end of the memory metal wire, and located outside the film sleeve;
the film sleeve has a length greater than a length of the deformation control unit core body wrapped inside the film sleeve;
an inner diameter of the film sleeve is greater than an outer diameter of the deformation control unit core body;
the film sleeve is made of an insulating, flame-retardant material;
an elastic module of the memory metal wire is not lower than that of the conductive metal wire;
the memory metal wire is made of a memory metal that is elastically deformed as a temperature changes or a current changes, and a radian of the memory metal wire is elastically deformed as the temperature changes or the current changes;
the aerogel particles are made of a low-density solid heat-insulating material;
the head end of the needle body sleeve extends into the minimally invasive surgical wound during surgery, and the length of the needle body sleeve satisfies that the tail end of the needle body sleeve is always located outside the minimally invasive surgical wound during surgery;
the electrical loop metal wire is located inside the needle body sleeve and outside the suture line sleeve; and
the lengths of the memory metal wire, the conductive metal wire and the electrical loop metal wire satisfy that the tail end of the conductive metal wire and the head end of the electrical loop metal wire are always located outside the minimally invasive surgical wound during surgery.

Preferably, a friction coefficient and an elastic modulus of the film sleeve satisfy that the strain of the film sleeve in a length direction is lower than 5% of the strain of the deformation control unit core body in a length direction when the deformation control unit core body in the film sleeve is bent, stretched or contracted.

Preferably, the radians of the conductive metal wire and the electrical loop metal wire do not change as the temperature changes or the current changes, and side surfaces of the conductive metal wire and the electrical loop metal wire are coated with an insulating coating.

Preferably, a largest Feret diameter of the aerogel particles is less than 0.1 mm.

Preferably, the arrangement of the control units and the line feeding unit in a flexible deformation needle body composed of parallel units with position differences also satisfies a minimum section diameter at the section of the maximum diameter of a flexible deformation needle body composed of parallel units with position differences.

Preferably,
when I=1 and the quantity of the line feeding unit is 1, the deformation control unit is adjacent to the line feeding unit at the cross section of the needle body;
when I=2 and the quantity of the line feeding unit is 1, the two deformation control units are adjacent to each other at the cross section of the needle body and both adjacent to the line feeding unit; and
when I≥3 and the quantity of the line feeding unit is 1, the deformation control units are all located on the outer side of the line feeding unit at the cross section of the needle body.

Preferably, the control units and the line feeding unit in the flexible deformation needle body composed of parallel units with position differences also satisfy:

$$\vert l - L , ( 1 ) \vert \times 10 \leq L , \qquad ( 1 )$$

wherein l represents a length of the suture line sleeve extending into the minimally invasive surgical wound.

The present invention further provides a form control method of a modular deformable suture needle for a minimally invasive surgery, which, based on the above-mentioned modular deformable suture needle for the minimally invasive surgery, includes the following steps:
obtaining a quantitative relationship between bending deformation features of each deformation control unit and a heat power provided by the corresponding heat supply unit or a voltage provided by the corresponding power supply unit through a statistical learning model;
implementing turning control on the corresponding memory metal wire by rotating the tail end of the deformation control unit and adjusting the corresponding heat power or voltage;
implementing coherent form control to move forward by controlling the voltages or heat powers of the deformation control units in an order of i increment or by rotating the tail end of the deformation control unit; and
implementing coherent form control to move backward by controlling the voltages or heat powers of the deformation control units in an order of i decrement or by rotating the tail end of the deformation control unit; wherein
preferably, the statistical learning model is a nonlinear function with voltage-related features or heat power-related features as independent variables and bending deformation features of the deformation control units as dependent variables.

Preferably,
the bending deformation features of the i^{th} deformation control unit include an arc-chord ratio αi, a peak deviation position βi, and are mouth directions, the are mouth directions including a first direction φ′δiζi and a second direction θi′δiζi, wherein
a central axis of the i^{th} deformation control unit is divided into a deformable segment and a non-deformable segment according to a boundary between the memory metal wire and the conductive metal wire of the i^{th} deformation control unit;
a space rectangular coordinate system oi-xiyizi is defined: a normal plane of the non-deformable segment on the central axis of the i^{th} deformation control unit at the boundary between the deformable segment and the non-deformable segment is taken as a coordinate plane zi-oi-yi, the central axis of the i^{th} deformation control unit passes through an origin oi, and a normal vector of the coordinate plane zi-oi-yi passing through the origin oi is taken as an xi axis;
a spherical coordinate system is defined as oi-riφiθi, and a conversion relationship between the two coordinate systems is as follows: coordinates of a point A in the rectangular coordinate system are set as (xi, yi, zi), spherical coordinates of the point A are set as (ri, φi, θi), the coordinate ri is a distance from the point A to the origin oi, and φi is an included angle formed between a half-plane passing through zi axis and point A and the coordinate plane zi-oi-xi; θi is an included angle between a line segment oiA and a positive direction of the zi axis;
the origin oi is a tail end point of the deformable segment on the central axis of the i^{th} deformation control unit; a farthest point on the central axis of the i^{th} deformation control unit from the origin oi is defined as a head end point gi of the deformable segment on the central axis of the it deformation control unit; a distance between gi and oi along the central axis of the i^{th} deformation control unit is a length of the deformable segment on the central axis of the i^{th} deformation control unit; a midpoint of the deformable segment on the central axis of the i^{th} deformation control unit is denoted as mi;

$$α i = \frac{L g ioi}{l g ioi}$$

wherein Lgioi represents the length of the deformable segment on the central axis of the i^{th} deformation control unit;
lgioi is a length of a straight segment gioi;

$$β i = \frac{2 η i \times γ i}{Lgioi}$$

$$η i = \{ \begin{matrix}1 , lmioi > lmigi \\ - 1 , lmioi < lmigi\end{matrix}$$

wherein γi is the shortest distance from mi to an intermediate surface;
the intermediate surface is a normal plane of the straight segment gioi passing through ζi;
ζi is a midpoint of the straight segment gioi;
ηi is a parameter of deflection;
lmioi is a length of a straight segment mioi;
lmigi is a length of a straight segment migi;

$$φ i {}^{′} δ i ζ i = φ i {}^{′} δ i ζ {i}^{′} v / 1 rad$$

$$θ i {}^{′} δ i ζ i = θ i {}^{′} δ i ζ {i}^{′} v / 1 rad$$

wherein spherical coordinates of a vector δiζi are (ri′δiζi′v, φi′δiζi′v, θi′δiζi′v);
δi is an arc bottom point, specifically an intersection point of the intermediate surface and the deformable segment on the central axis of the i^{th} deformation control unit; and
a part of the modular deformable suture needle for the minimally invasive surgery extending into the wound being able to smoothly turn, move forward and move backward or make other actions when a region to be sutured is blocked and covered by tissues and organs is taken as a precondition, and the workload in the coherent form control is minimum, that is, the lowest overall bending deformation degree Σαi of the part of the modular deformable suture needle for the minimally invasive surgery extending into the wound is taken as a control object, and Σζi is a sum of αi of the deformation control units where all memory metal wires extending into the minimally invasive surgical wound are located.

The present invention has the following beneficial effects.

The needle body of the modular deformable suture needle for the minimally invasive surgery is formed by differential parallel connection of the tough deformation control units, and has the advantages of controllable deformation of each segment and strong axial toughness-integrity of the needle body at the same time, and overcomes the limitations existing in the prior art:
(1) the structure diameter is small, a parallel connection structure has strong axial toughness-integrity, and avoids the disadvantage that a series-assembled structure is vulnerable to fracture, and apparatuses have high safety;
(2) the dynamic deformations can be quantitatively controlled in the body of the surgical subject through an external voltage or heat power input in the suturing process, and there is no need to repeatedly remove a suturing apparatus from the body and then back to the body of the surgical subject, so the suturing efficiency is high; and
(3) the form control method of the modular deformable suture needle for the minimally invasive surgery can quantitatively control independent controllable continuous deformations of the needle body segments in all degrees of freedom and all directions, so that the suture needle can smoothly turn, move forward, and move backward or make other actions when the region to be sutured is blocked and covered by tissues and organs, and the device has strong deformability.

### BRIEF DESCRIPTION OF THE DRAWINGS

FIG. 1 is a structural diagram of a deformation control unit core body;

FIG. 2 is a diagram of axial differential structure of a deformation control unit cluster;

FIG. 3 is a diagram of a flexible deformation needle body composed of parallel units with position differences in a straight state;

FIG. 4 is a schematic diagram of coordinate system conversion of an i^{th} deformation control unit;

FIG. 5 is a schematic diagram of segmented deformations of the i^{th} deformation control unit;

FIG. 6 shows a maximum cross section of the flexible deformation needle body composed of parallel units with position differences when I=1;

FIG. 7 is a front view of a modular deformable suture needle for a minimally invasive surgery in Embodiment 2;

FIG. 8 is a side view of the modular deformable suture needle for the minimally invasive surgery in Embodiment 2;

FIG. 9 shows a maximum cross section of the flexible deformation needle body composed of parallel units with position differences when I=2;

FIG. 10 is a front view of a modular deformable suture needle for a minimally invasive surgery in Embodiment 3, from which a needle body sleeve is hidden;

FIG. 11 is a side view of the modular deformable suture needle for the minimally invasive surgery in Embodiment 3;

FIG. 12 is a front view of a modular deformable suture needle for a minimally invasive surgery in Embodiment 4, from which a needle body sleeve is hidden;

FIG. 13 is a side view of the modular deformable suture needle for the minimally invasive surgery in Embodiment 4;

FIG. 14 shows a maximum cross section of the flexible deformation needle body composed of parallel units with position differences in Embodiment 4;

FIG. 15 is a bending deformation diagram of each deformation control unit in a needle body sleeve in Embodiment 5; and

FIG. 16 shows a maximum cross section of the flexible deformation needle body composed of parallel units with position differences in Embodiment 5.

Reference symbols represent the following components: 1—memory metal wire; 2—conductive metal wire; 3—aerogel particles; 4—film sleeve; 5—deformation control unit; 6—suture line; 7—suture line sleeve; 8—line feeding unit; 9—needle body sleeve; 10—arc—shaped needle; 11—straight needle; and 15—electrical loop metal wire.

### DETAILED DESCRIPTION OF THE INVENTION

In order to make the purposes, features, and advantages of the present invention more obvious and understandable, the technical solutions in the embodiments of the present invention will be described clearly and completely in conjunction with the accompanying drawings in the embodiments of the present invention. Apparently, the described embodiments are merely some embodiments, rather than all embodiments, of the present invention. Based on the embodiments of the present disclosure, all other embodiments derived by a person of ordinary skill in the art without creative efforts shall fall within the protection scope of the present disclosure.

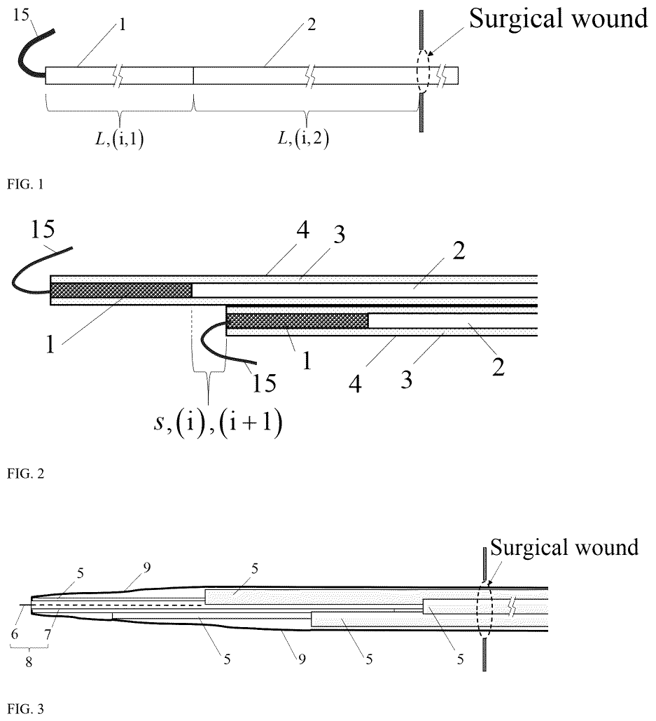

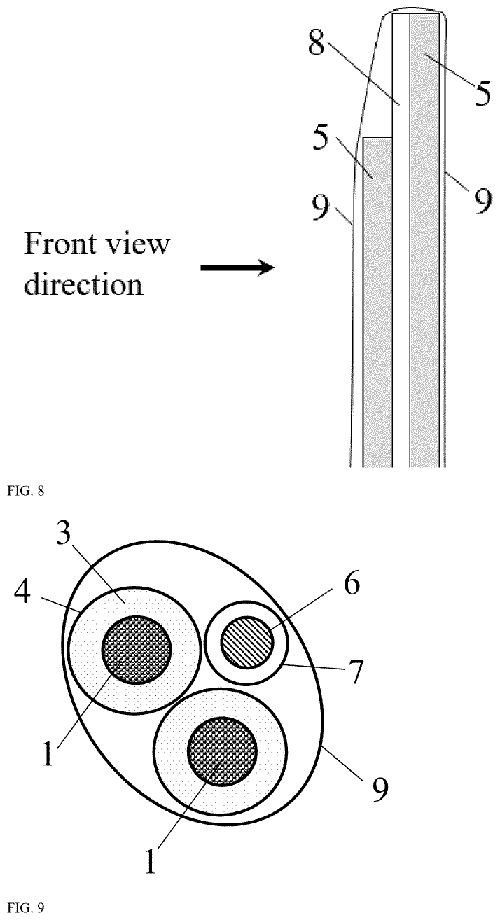

As shown in FIG. 1 to FIG. 16, through the following steps, a construction process and a control method of the product are introduced.

Step 1: Constructing a Flexible Deformation Needle Body Composed of Parallel Units with Position Differences

### (1) Structure of a Single Deformation Control Unit 5

The single deformation control unit 5 includes a memory metal wire 1, a conductive metal wire 2, aerogel particles 3, a film sleeve 4, and an electrical loop metal wire 15, wherein
a tail end of the memory metal wire 1 is electrically connected to a head end of the conductive metal wire 2 to form a deformation control unit core body of the single deformation control unit 5, a head end of the deformation control unit core body is a head end of the memory metal wire 1, and a tail end of the deformation control unit core body is a tail end of the conductive metal wire 2;
the film sleeve 4 wraps the memory metal wire 1 and the conductive metal wire 2, one end of the film sleeve 4 closest to the head end of the memory metal wire 1 is a head end of the film sleeve 4, and the other end of the film sleeve 4 except the head end is a tail end of the film sleeve 4;
the aerogel particles 3 are filled between the film sleeve 4 and the wrapped memory metal wire 1 and conductive metal wire 2;
a tail end of the electrical loop metal wire 15 is electrically connected to the head end of the memory metal wire 1, and located outside the film sleeve 4;
the film sleeve 4 has a length greater than a length of the deformation control unit core body wrapped inside the film sleeve;
an inner diameter of the film sleeve 4 is greater than an outer diameter of the deformation control unit core body;
the film sleeve 4 is made of an insulating, flame-retardant material;
a friction coefficient and an elastic modulus of the film sleeve 4 satisfy that the strain of the film sleeve 4 in a length direction is lower than 5% of the strain of the deformation control unit core body in a length direction when the deformation control unit core body in the film sleeve 4 is bent, stretched or contracted;
an elastic module of the memory metal wire 1 is not lower than that of the conductive metal wire 2;
the memory metal wire 1 is made of a memory metal that is elastically deformed as a temperature changes or a current changes, and a radian of the memory metal wire 1 is elastically deformed as the temperature changes or the current changes;
the radians of the conductive metal wire 2 and the electrical loop metal wire 15 do not change as the temperature changes or the current changes, and side surfaces of the conductive metal wire 2 and the electrical loop metal wire 15 are coated with an insulating coating;
a largest Feret diameter of the aerogel particles 3 is less than 0.1 mm; and
the aerogel particles 3 are a low-density solid heat-insulating material, and compared with inert gases, oils, foam plastics and rubber, reduces the mass of a filling body while effectively insulating heat, is not prone to leakage and dissipation, reduces a load of the deformation control unit core body, and improves the deformation control accuracy of the deformation control units.

### (2) Line Feeding Units

A single line feeding unit 8 includes a suture line 6 and a suture line sleeve 7, wherein the suture line sleeve 7 wraps the suture line 6;
an inner diameter of the suture line sleeve 7 is greater than an outer diameter of the suture line 6; and an elastic modulus of the suture line sleeve 7 is lower than that of the film sleeve 4.

### (3) Needle Body Structure

The flexible deformation needle body composed of parallel units with position differences contains deformation control units 5 and line feeding units 8 inside;
the quantity I of deformation control units 5 where the memory metal wire 1 is located extending into a minimally invasive surgical wound is greater than or equal to 1 in the single flexible deformation needle body composed of parallel units with position differences, and the serial number of the deformation control unit in the needle body is i, 1≤i≤I;
the quantity of the line feeding units 8 is not less than 1; and
the arrangement of the control units 5 and the line feeding units 8 in the flexible deformation needle body composed of parallel units with position differences also satisfies a minimum section diameter at the section of the maximum diameter of the flexible deformation needle body composed of parallel units with position differences, which ensures that the Feret diameter of a pore that the flexible deformation needle body composed of parallel units with position differences can pass through is minimum and thus widest application range, which are of the most fundamental principle of the needle body structure.

### 1) Cross Section Structure

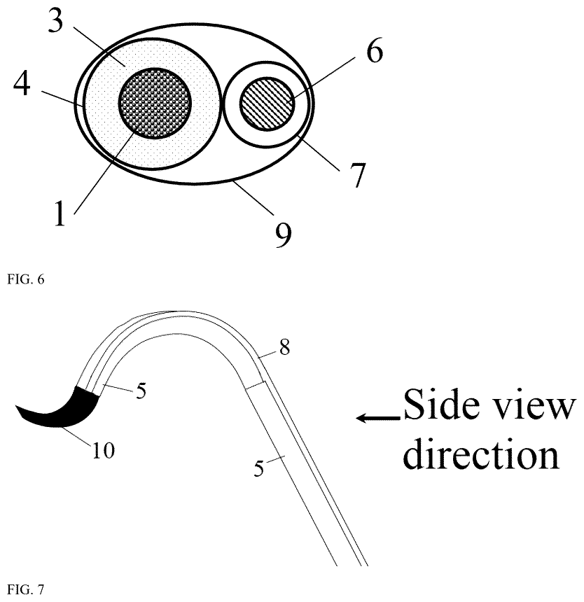

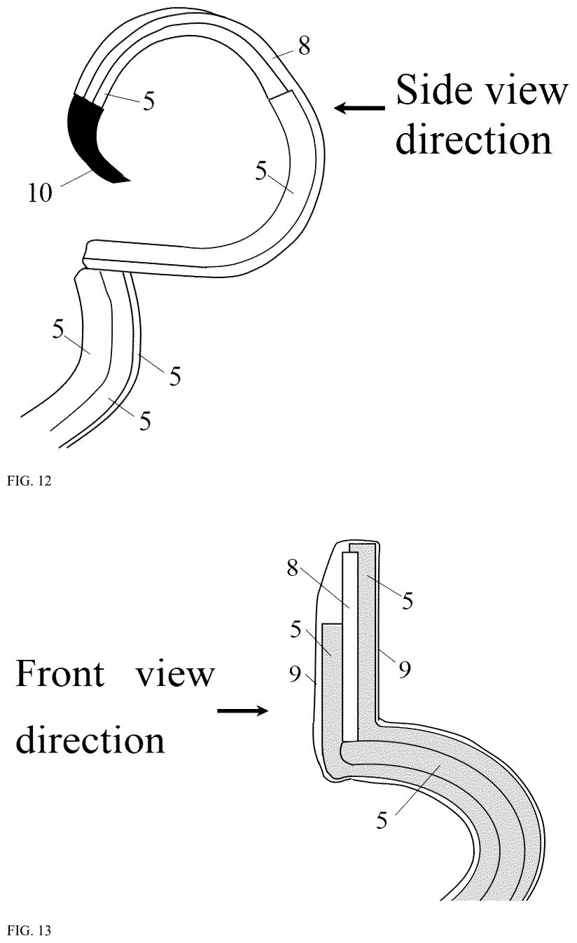

In order to reduce position control errors of the suture needle and the head end of the suture line, a distance between a central axis of the needle body sleeve 9 and a central axis of the suture line sleeve 7 should be minimized under the premise of ensuring the minimum section diameter at the section of the maximum diameter of the flexible deformation needle body composed of parallel units with position differences, so that a spatial position of the head end of the suture line 6 is closest to a spatial position of the head end of the flexible deformation needle body composed of parallel units with position differences, thereby minimizing the control errors. The corresponding cross section structure is as follows:
as shown in FIG. 6, when I=1, the deformation control unit and the line feeding unit at the cross section of the needle body are adjacent and both located inside the needle body sleeve 9 [Embodiment 1];
as shown in FIG. 7 to FIG. 11, when I=2, the two deformation control units 5 are adjacent to each other at the cross section of the needle body, and both adjacent to the line feeding unit 8, and the deformation control units and the line feeding unit are all located inside the needle body sleeve 9 [Embodiment 2 and Embodiment 3]; and
as shown in FIG. 12 to FIG. 16, when I≥3, the deformation control units 5 are all located outside the line feeding unit and inside the needle body sleeve 9 at the cross section of the needle body [Embodiment 4 and Embodiment 5].

### 2) Axial Differential Structure of a Deformation Control Unit Cluster

A length of the i^{th} deformation control unit 5 extending into the minimally invasive surgical wound is L,(i), wherein L,(i) is equal to a sum of a length L,(i,1) of the memory metal wire extending into the minimally invasive surgical wound and a length L,(i,2) of the conductive metal wire extending into the minimally invasive surgical wound, L,(i)>L,(i+1), L,(1)>L,(2)>L,(3)> . . . >L,(I).

A combined differential structure of the deformation control units is shown in Equation (1), wherein s,(i),(i+1) represents a distance between a tail end of the memory metal wire 1 in the i^{th} deformation control unit 5 and a head end of the memory metal wire 1 in the (i+1)^{th} deformation control unit 5.

s,(i),(i+1)>0 ensures that the deformation of the memory metal wire 1 in each deformation control unit is independent of each other and does not interfere with each other, so that a part of the flexible deformation needle body composed of parallel units with position differences extending into the wound can undergo bending-straightening deformations in multiple degrees of freedom and multiple directions in segments, so the segments of the whole needle body have controllable and continuous deformation capacities in all degrees of freedom and all directions.

If the length of the memory metal wire 1 extending into the minimally invasive surgical wound is greater than zero, the deformation control unit 5 where the memory metal wire 1 is located is the deformation control unit 5 where the memory metal wire 1 extending into the minimally invasive surgical wound is located,

$$s , ( i ) , ( i + 1 ) = L , ( i ) - L , ( i + 1 ) - L , ( i , 1 ) . \qquad ( 1 )$$

### 3) Line Feeding Structure

In order to reduce a stiffness difference between the suture line sleeve 7 and the deformation control unit 5, improve the synchronous coordination of deformations between the suture line sleeve 7 and the deformation control unit 5, reduce a difference in a spatial position between the head end of the suture line sleeve 7 and the head end of the first deformation control unit 5, improve the deformation control accuracy, and reduce the area of a gap between an outer edge of the head end of the suture line sleeve 7 and the head end of the film sleeve 4 of the first deformation control unit, so as to improve the sealing reliability of the needle body sleeve 9, the length l of the suture line sleeve 7 extending into the minimally invasive surgical wound should satisfy Equation (2),

$$\vert l - L , ( 1 ) \vert \times 10 \leq L , ( 1 ) . \qquad ( 2 )$$

### 4) Sealing Measures

The needle body sleeve 9 is made of an antibacterial elastic material, wraps the deformation control unit 5 and the line feeding unit 8 that are inserted into the body of the surgical subject, and plays the role of isolating the deformation control unit 5 and the line feeding unit 8 from organ tissues in the body of the surgical subject.

The head end of the needle body sleeve 9 is connected to the outer edge of the head end of the suture line sleeve 7 in a fixed sealing manner, and a part of each deformation control unit 5 extending into the wound is isolated from organ tissues in the body of the surgical subject by the needle body sleeve 9, so fluid in surgery cannot penetrate into the needle body sleeve 9.

The head end of the needle body sleeve 9 extends into the minimally invasive surgical wound during surgery. In order to ensure the effective isolation of the needle body sleeve 9 for the organ tissues in the body of the surgical subject 9 from the deformation control unit and the line feeding unit, the length of the needle body sleeve 9 should ensure that the tail end of the needle body sleeve 9 is always located outside the minimally invasive surgical wound during surgery.

The electrical loop metal wire 15 is located inside the needle body sleeve 9 and outside the suture line sleeve 7.

The lengths of the memory metal wire 1, the conductive metal wire 2 and the electrical loop metal wire 15 should ensure that the tail end of the conductive metal wire 2 and the head end of the electrical loop metal wire 15 are always located outside the minimally invasive surgical wound during surgery.

### Step 2: Constructing Modular Deformable Suture Needle for Minimally Invasive Surgery

The modular deformable suture needle for the minimally invasive surgery includes a short needle, a flexible deformation needle body composed of parallel units with position differences, and a heat supply unit or a power supply unit.

The short needle is an arc-shaped needle 10 or a straight needle 11.

A head end of the arc-shaped needle 10 and a head end of the straight needle 11 are needle tips, and a tail end of the arc-shaped needle 10 and a tail end of the straight needle 11 are connected to the suture line 6.

A heat source is connected in series with a switch to form the heat supply unit of the single deformation control unit 5.

A power source is connected in series with the switch to form the power supply unit of the single deformation control unit 5.

Any pole in positive and negative poles of the power source is electrically connected to the tail end of the conductive metal wire 2 through the switch, and the other pole is electrically connected to the head end of electrical loop metal wire 15, so that the power supply unit of the single deformation control unit 5 is connected in series with the single deformation control unit 5.

The heat source is connected to the tail end of the conductive metal wire 2 through the switch in a heat conduction manner, such that the heat supply unit of the single deformation control unit 5 is connected in series with the single deformation control unit 5.

### Step 3: Quantitatively Expressing Bending Deformation Features

This step is directed to a case that the single deformation control unit 5 is always in no contact with other deformation control units.

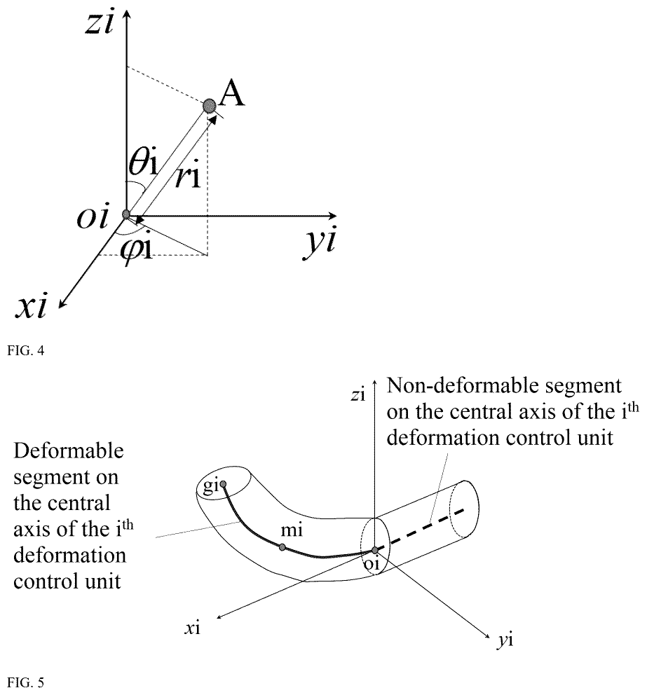

As shown in FIG. 4 to FIG. 5, a space rectangular coordinate system and a spherical coordinate system of the i^{th} deformation control unit are defined as oi-xiyizi and oi-riφiθi, and a conversion relationship between the two coordinate systems is as follows: coordinates of a point A in the rectangular coordinate system are set as (xi, yi, zi), spherical coordinates of the point A are set as (ri, φi, θi), the coordinate ri is a distance from the point A to the origin oi, and φi is an included angle formed between the half-plane passing through zi axis and point A and the coordinate plane zi-oi-xi; and θi is an included angle between a line segment oiA and a positive direction of the zi axis.

A central axis of the i^{th} deformation control unit is divided into a deformable segment and a non-deformable segment according to a boundary between the memory metal wire 1 and the conductive metal wire 2 of the i^{th} deformation control unit 5.

The space rectangular coordinate system is defined as oi-xiyizi: a normal plane of the non-deformable segment on the central axis of the i^{th} deformation control unit 5 at the boundary between the deformable segment and the non-deformable segment is taken as a coordinate plane zi-oi-yi, the central axis of the i^{th} deformation control unit 5 passes through an origin oi, and a normal vector of the coordinate plane zi-oi-yi passing through the origin oi is taken as an xi axis.

The origin oi is a tail end point of the deformable segment on the central axis of the i^{th} deformation control unit 5; a farthest point on the central axis of the i^{th} deformation control unit 5 from the origin oi is defined as a head end point gi of the deformable segment on the central axis of the i^{th} deformation control unit 5; a distance between gi and oi along the central axis of the i^{th} deformation control unit 5 is a length of the deformable segment on the central axis of the i^{th} deformation control unit; and a midpoint of the deformable segment on the central axis of the i^{th} deformation control unit is mi.

According to a conversion relationship between the space rectangular coordinate system oi-xiyizi and the spherical coordinate system oi-riφiθi, the coordinates of gi, mi, and oi in the spherical coordinate system oi-riφiθi are (ri′gi, φi′gi, θi′gi), (ri′mi, φi′mi, θi′mi), (0,0,0), respectively.

The bending deformation features of the i^{th} deformation control unit include an arc-chord ratio αi, a peak deviation position βi, and are mouth directions.

The arc-chord ratio αi is a ratio of the length Lgioi of the deformable segment on the central axis of the i^{th} deformation control unit 5 to a chord length lgioi of the i^{th} deformable segment, which reflects the deformation degree of the memory metal; and lgioi is a length of a straight segment gioi, as shown in Equation (3),

$$α i = \frac{L g ioi}{l g ioi} . \qquad ( 3 )$$

The peak deviation position βi reflects a relative positional relationship between mi and gi as well as oi. As shown in Equation (4), γi is the shortest distance from mi to the intermediate surface; the intermediate surface is a normal plane of the straight segment gioi passing through a midpoint ζi of the straight segment gioi; lmioi is the length of the straight segment mioi; lmigi is the length of the straight segment migi; and the parameter of deflection ηi is shown in Equation (5),

$$β i = \frac{2 η i \times γ i}{Lgioi} \qquad ( 4 )$$

$$η i = \{ \begin{matrix}1 , lmioi > lmigi \\ - 1 , lmioi < lmigi\end{matrix} . \qquad ( 5 )$$

The are mouth directions include a first direction φi′δiζi=φi′Siζi′v/1 rad and a second direction θi′δiζi=θi′δiζi′v/1 rad. An intersection point of the intermediate surface and the deformable segment on the central axis of the i^{th} deformation control unit 5 is defined as an arc bottom point δi, and then spherical coordinates of a vector δiζi are (ri′δiζi′v,φi′δiζi′v,θi′δiζi′v).

The reason why the non-deformable segment on the central axis of the i^{th} deformation control unit 5 cannot undergo bending-straightening deformations is not that the material stiffness is infinite, but that the non-deformable segment of the i^{th} deformation control unit 5 cannot undergo bending-straightening deformations due to a voltage or heating under the condition that the i^{th} deformation control unit 5 is always in no contact with other deformation control units 5. The non-deformable segment of the i^{th} deformation control unit 5 and other deformation control units 5 that have been deformed can be driven to undergo corresponding bending-straightening deformations when they are in contact with each other.

### Step 4: Constructing a Single-Segment Form Control Model

This step is directed to a case that the single deformation control unit 5 is always in no contact with other deformation control units 5.

Deformation stabilization moment: an absolute value of a difference between the first direction of the deformation control unit 5 at the current moment and the first direction 10 seconds before the current moment does not exceed 5% of the first direction 10 seconds before the current moment, and then the current moment is the deformation stabilization moment.

### (1) Electrically Controlled Deformation Measurement

A j-level voltage of the power supply unit to which the i^{th} deformation control unit 5 is connected is Ui,j=j×Δui, wherein j is a voltage level, and a voltage level difference Δui is greater than or equal to 0.01 V.

After Ui,j is applied to the i^{th} deformation control unit 5, when the deformation control unit 5 is stabilized in deformation, an arc-chord ratio αi,j, a peak deviation position βi,j, a first direction φi′δiζi,j and a second direction θi′δiζi,j of the i^{th} deformation control unit that is bent under Ui,j are measured.

A measured sample of electronically controlled deformation is {(αi,j, βi,j, φi′δiζi,j, θi′δiζi,j, Ui,j)| 0<j≤J, j∈Z}, wherein J is the highest voltage level, J is greater than and equal to 100, and Z is a set of integers.

### (2) Thermally Controlled Deformation Measurement

A k-level heat power of the heat supply unit to which the i^{th} deformation control unit is applied is Pi,k=kxΔpi, wherein k is a heat power level, and a heat power level difference Δpi is greater than or equal to 0.1 W.

After Pi,k is applied to the i^{th} deformation control unit 5, when the deformation control unit is stabilized in deformation, an arc-chord ratio αi,k, a peak deviation position βi,k, a first direction (φi′δiζi,k and a second direction θi′δiζi,k of the i^{th} deformation control unit that is bent under Pi,k are measured.

A measured sample of thermally controlled deformation is {(αi,k, βi,k, φi′δiζi,k, θi′δiζi,k, Pi,k) | 0<k≤K, k∈Z}, wherein K is the highest heat power level, K is greater than and equal to 100, and Z is a set of integers.

### (3) Deformation Control Model

The deformation control model includes an electrically controlled deformation segmentation model and a thermally controlled deformation segmentation model.

When the memory metal wire is controlled to deform by applying a voltage, the electrically controlled deformation segmentation model is shown in Equation (6), j′=j−1.

\hat{α}i,j, \hat{β}i,j, \hat{φ}i′iζi,j and \hat{θ}i′δiζi,j are predicted values of αi,j, βi,j, φi′δiζi,j and θi′δiζi,j respectively; a voltage feature Ui is a ratio of a voltage value to 1 V.

a′1,j, a′2,j, a′3,j, a′4,j and a′5,j are coefficients of Ui^{1/3}, Ui^{2/3}, Ui, Ui^{4/3} and Ui² in a \hat{α}i,j prediction model, respectively.

b′1,j, b′2,j, b′3,j, b′4,j and b′5,j are coefficients of Ui^{1/3}, Ui^{2/3}, Ui, Ui^{4/3} and Ui² in \hat{β}i,j prediction model, respectively.

c′1,j, c′2,j, c′3,j, c′4,j and c′5,j are coefficients of Ui^{1/3}, Ui^{2/3}, Ui, Ui^{4/3} and Ui² in a \hat{φ}i′δiζi,j prediction model, respectively.

d′1,j, d′2,j, d′3,j, d′4,j and d′5,j are coefficients of Ui^{1/3}, Ui^{2/3}, Ui, Ui^{4/3} and Ui² in a \hat{θ}i′δiζi,j prediction model, respectively,

$$\{ \begin{matrix}\begin{matrix}\hat{α} i , j = 1 + ( {a}^{′} 1 , j ) {Ui}^{1 / 3} + ( {a}^{′} 2 , j ) {Ui}^{2 / 3} + ( {a}^{′} 3 , j ) Ui + ( {a}^{′} 4 , j ) {Ui}^{4 / 3} + \\ ( {a}^{′} 5 , j ) {Ui}^{2} , \frac{Ui , {j}^{′}}{1 V} \leq Ui < \frac{Ui , j}{1 V}\end{matrix} \\ \begin{matrix}\hat{β} i , j = ( {b}^{′} 1 , j ) {Ui}^{1 / 3} + ( {b}^{′} 2 , j ) {Ui}^{2 / 3} + ( {b}^{′} 3 , j ) Ui + ( {b}^{′} 4 , j ) {Ui}^{4 / 3} + \\ ( {b}^{′} 5 , j ) {Ui}^{2} , \frac{Ui , {j}^{′}}{1 V} \leq Ui < \frac{Ui , j}{1 V}\end{matrix} \\ \begin{matrix}\hat{φ} i {}^{′} δ i ζ i , j = ( {c}^{′} 1 , j ) {Ui}^{1 / 3} + ( {c}^{′} 2 , j ) {Ui}^{2 / 3} + ( {c}^{′} 3 , j ) Ui + ( {c}^{′} 4 , j ) {Ui}^{4 / 3} + \\ ( {c}^{′} 5 , j ) {Ui}^{2} , \frac{Ui , {j}^{′}}{1 V} \leq Ui < \frac{Ui , j}{1 V}\end{matrix} \\ \begin{matrix}\hat{θ} i {}^{′} δ i ζ i , j = ( {d}^{′} 1 , j ) {Ui}^{1 / 3} + ( {d}^{′} 2 , j ) {Ui}^{2 / 3} + ( {d}^{′} 3 , j ) Ui + ( {d}^{′} 4 , j ) {Ui}^{4 / 3} + \\ ( {d}^{′} 5 , j ) {Ui}^{2} , \frac{Ui , {j}^{′}}{1 V} \leq Ui < \frac{Ui , j}{1 V}\end{matrix}\end{matrix} . \qquad ( 6 )$$

When the memory metal wire is controlled to deform by applying heat conduction, the thermally controlled deformation segmentation model is shown in Equation (7), k′=k−1.

\hat{α}i,k, \hat{β}i,k, \hat{φ}i′δiζi,k and \hat{θ}i′δiζi,k are predicted values of αi,k, βi,k, φi′δiζi,k and θi′δiζi,k, respectively; a heat power feature Pi is a ratio of a heat power to 1 W.

e′1,k,′, e′2,k, e′3,k, e′4,k and e′5,k are coefficients of Pi^{1/3}, Pi^{2/3}, Pi, pi^{4/3} and Pi^{1.5} in a \hat{α}i,k prediction model, respectively.

f′1,k, f′2,k, f′3,k, f′4,k and f′5,k are coefficients of Pi^{1/3}, Pi^{2/3}, Pi, Pi^{4/3} and Pi^{1.5} in \hat{β}i,k prediction model, respectively.

λ′1,k, λ′2,k, λ′3,k, λ′4,k and λ′5,k are coefficients of Pi^{1/3}, Pi^{2/3}, Pi, Pi^{4/3} and Pi^{1.5} in a \hat{φ}i′δiζi,k prediction model, respectively.

τ′1,k, τ′2,k, τ′3,k, τ′4,k and τ′5,k are coefficients of Pi^{1/3}, Pi^{2/3}, Pi, Pi^{4/3} and Pi^{1.5} in a \hat{θ}i′δiζi,k prediction model, respectively.

$$\{ \begin{matrix}\begin{matrix}\hat{α} i , k = 1 + ( {e}^{′} 1 , k ) {Pi}^{1 / 3} + ( {e}^{′} 2 , k ) {Pi}^{2 / 3} + ( {e}^{′} 3 , k ) Pi + ( {e}^{′} 4 , k ) {Pi}^{4 / 3} + \\ ( {e}^{′} 5 , k ) {Pi}^{1.5} , \frac{Pi , {k}^{′}}{1 W} \leq Pi < \frac{Pi , k}{1 W}\end{matrix} \\ \begin{matrix}\hat{β} i , k = ( f 1 , k ) {Pi}^{1 / 3} + ( f 2 , k ) {Pi}^{2 / 3} + ( f 3 , k ) Pi + ( f 4 , k ) {Pi}^{4 / 3} + \\ ( f 5 , k ) {Pi}^{1.5} , \frac{Pi , {k}^{′}}{1 W} \leq Pi < \frac{Pi , k}{1 W}\end{matrix} \\ \begin{matrix}\hat{φ} i {}^{′} δ i ζ i , k = ( {λ}^{′} 1 , k ) {Pi}^{1 / 3} + ( {λ}^{′} 2 , k ) {Pi}^{2 / 3} + ( {λ}^{′} 3 , k ) Pi + ( {λ}^{′} 4 , k ) {Pi}^{4 / 3} + \\ ( {λ}^{′} 5 , k ) {Pi}^{1.5} , \frac{Pi , {k}^{′}}{1 W} \leq Pi < \frac{Pi , k}{1 W}\end{matrix} \\ \begin{matrix}\hat{θ} i {}^{′} δ i ζ i , k = ( {τ}^{′} 1 , k ) {Pi}^{1 / 3} + ( {τ}^{′} 2 , k ) {Pi}^{2 / 3} + ( {τ}^{′} 3 , k ) Pi + ( {τ}^{′} 4 , k ) {Pi}^{4 / 3} + \\ ( {τ}^{′} 5 , k ) {Pi}^{1.5} , \frac{Pi , {k}^{′}}{1 W} \leq Pi < \frac{Pi , k}{1 W}\end{matrix}\end{matrix} . \qquad ( 7 )$$

Equation (6) and Equation (7) are respectively used to fit the measured sample of electronically controlled deformation and the measured sample of thermal control deformation: the coefficients in Equation (6) and Equation (7) are obtained by using a least squares method, so that the arc-chord ratio, the peak deviation position and the are mouth directions are explicitly predicted by Equation (6) and Equation (7) respectively under a given voltage or heat power, so as to control the deformation of the single deformation control unit by adjusting the voltage or heat power.

### Step 5: Coherent Form Control

When the deformable segment on the central axis of the (i+1)^{th} deformation control unit 5 undergoes a bending-straightening deformation, the non-deformable segment on the central axis of the i^{th} deformation control unit 5 deforms following the deformable segment on the central axis of the (i+1)^{th} deformation control unit 5 under the drive of the deformable segment on the central axis of the (i+1)^{th} deformation control unit 5.

A part of the modular deformable suture needle for the minimally invasive surgery extending into the wound is able to smoothly turn, move forward, move backward or make other actions when a region to be sutured being blocked and covered by tissues and organs is taken as a precondition, and the workload in the coherent form control is minimum, that is, the lowest overall bending deformation degree Σαi of the part of the modular deformable suture needle for the minimally invasive surgery is taken as a control object, and Σαi is a sum of αi of the deformation control units where all memory metal wires extending into the minimally invasive surgical wound are located.

### (1) Turning Control Method

The deformation control unit 5 in the first direction is roughly adjusted by rotating the tail end of the single deformation control unit 5. According to step 4, the deformation control unit 5 in the first direction and the second direction are finely adjusted by adjusting the voltage or heat power.

### (2) Method of Coherent Form Control to Move Forward

On the premise of ensuring that the modular deformable suture needle for the minimally invasive surgery penetrates a passable gap in organs tissues in the body of the surgical subject, the arc-chord ratio and the peak deviation position of each deformation control unit are finely adjusted by adjusting the voltage or heat power in step 4 according to the order of i increment; and the are mouth directions of each deformation control unit are roughly adjusted and finely adjusted according to the turning control method, so as to realize that the modular deformable suture needle for the minimally invasive surgery is inserted into the wound and advances to the region to be sutured.

### (3) Method of Coherent Form Control to Move Backward

On the premise of ensuring that the modular deformable suture needle for the minimally invasive surgery penetrates a passable gap in organs tissues in the body of the surgical subject, the arc-chord ratio and the peak deviation position of each deformation control unit are finely adjusted by adjusting the voltage or heat power in step 4 according to the order of i decrement; and the are mouth directions of each deformation control unit are roughly adjusted and finely adjusted according to the turning control method, so as to realize that the modular deformable suture needle for the minimally invasive surgery moves backward from the region to be sutured to the wound.

### Embodiment 1

When I=1, the deformation control unit and the line feeding unit are adjacent to each other at the cross section of the needle body and both located inside the needle body sleeve 9; and relative positions of the deformation control unit and the line feeding unit at the maximum cross section of the flexible deformation needle body composed of parallel units with position differences are shown in FIG. 6.

### Embodiment 2

When I=2, the two deformation control units 5 are adjacent to each other at the cross section of the needle body and both adjacent to the line feeding unit 8, and the deformation control units and the line feeding unit are all located inside the needle body sleeve 9.

### Embodiment 3

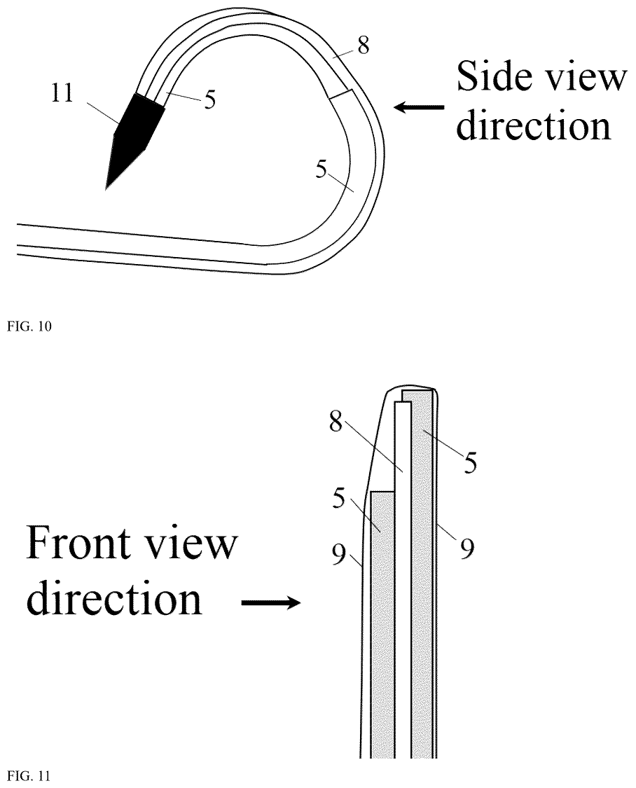

When I=2, the two deformation control units 5 are adjacent to each other at the cross section of the needle body and both adjacent to the line feeding unit 8, and the deformation control units and the line feeding unit are all located inside the needle body sleeve 9. In addition, when the first deformation control unit 5 and the second deformation control unit 5 undergo a bending deformation, and φ1′δ1ζ1′v=φ2′δ2ζ2′v, and an included angle between a vector δ1ζ1 and a vector δ2ζ2 is an obtuse angle, the front and side views of the modular deformable suture needle for the minimally invasive surgery are FIG. 10 and FIG. 11, respectively.

### Embodiment 4

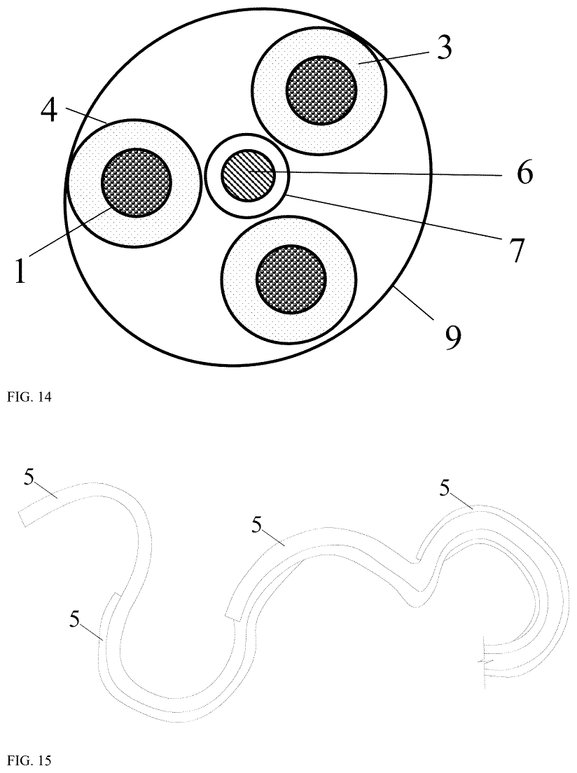

When I=3, the first deformation control unit, the second deformation control unit, and the third deformation control unit are all subjected to a bending deformation, and φ1′δ1ζ1′v=φ2′δ2ζ2′v≠φ3′δ3ζ3′v, an included angle between the vector δ1ζ1 and the vector δ2ζ2 is an obtuse angle, and an included angle between a vector δ3ζ3 and a vector δ2ζ2 is an obtuse angle, the front and side views of the modular deformable suture needle for the minimally invasive surgery are FIG. 12 and FIG. 13, respectively; and relative positions of the deformation control units and the line feeding unit at the maximum cross section of the flexible deformation needle body composed of parallel units with position differences is shown in FIG. 14.

### Embodiment 5

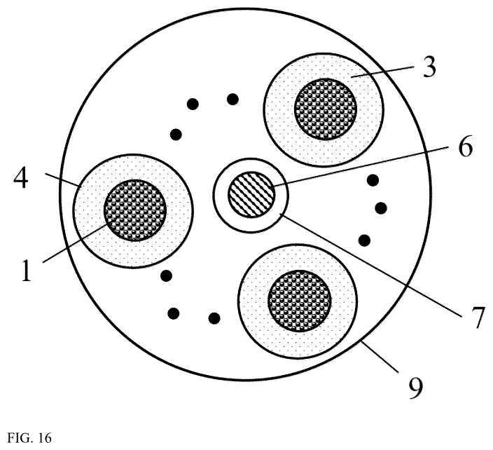

As shown in FIG. 15, I>3, each deformation control unit in the needle body sleeve 9 undergoes a bending deformation, and the non-deformable segment on the central axis of the i^{th} deformation control unit deforms following the deformable segment on the central axis of the (i+1)th deformation control unit under the drive of the deformable segment on the central axis of the (i+1)^{th} deformation control unit.

The relative positions of the deformation control units and the line feeding unit at the maximum cross section of the flexible deformation needle body composed of parallel units with position differences is shown in FIG. 16.

The above description is only preferred embodiments of the present invention, and it should be noted that those of ordinary skill in the art may also make several improvements and modifications without departing from the principles of the present invention, which should be considered as the protection scope of the present invention.

1. A modular deformable suture needle for a minimally invasive surgery, characterized by comprising a short needle, a flexible deformation needle body composed of parallel units with position differences, and a heat supply unit or a power supply unit, wherein
the flexible deformation needle body composed of parallel units with position differences comprises a needle body sleeve, I deformation control units, and more than one line feeding unit, in which, I≥1, and if the serial number of the deformation control unit is i, 1≤i≤I;
the line feeding unit comprises a suture line and a suture line sleeve, wherein the suture line sleeve wraps the suture line;
each deformation control unit comprises a memory metal wire, a conductive metal wire, aerogel particles and a film sleeve, wherein the tail end of the memory metal wire is sequentially connected to the head end of the conductive metal wire to form a deformation control unit core body, the film sleeve is wrapped outside the deformation control unit core body, and the aerogel particles are filled between the film sleeve and the deformation control unit core body; when the modular deformable suture needle for the minimally invasive surgery comprises the power supply unit, the deformation control unit further comprises an electrical loop metal wire, and a tail end of the electrical loop metal wire is connected to a head end of the memory metal wire;
the needle body sleeve is wrapped outside the deformation control units and the line feeding unit, the electrical loop metal wire is located inside the needle body sleeve and outside the film sleeve, and a head end of the electrical loop metal wire extends to a tail end of the needle body sleeve;
the arrangement of the deformation control units in the needle body sleeve satisfies:
$$L , ( i ) > L , ( i + 1 ) ;$$
$$s , ( i ) , ( i + 1 ) > 0 ;$$
$$s , ( i ) , ( i + 1 ) = L , ( i ) - L , ( i + 1 ) - L , ( i , 1 ) ;$$
wherein L,(i) represents a length of the i^{th} deformation control unit extending into a minimally invasive surgical wound, having a value equal to a sum of a length of the memory metal wire of the i^{th} deformation control unit extending into the minimally invasive surgical wound and a length of the conductive metal wire of the i^{th} deformation control unit extending into the minimally invasive surgical wound;
L,(i,1) represents the length of the memory metal wire of the i^{th} deformation control unit extending into the minimally invasive surgical wound;
s,(i),(i+1) represents a distance between a tail end of the memory metal wire in the i^{th} deformation control unit and a head end of the memory metal wire in the (i+1)^{th} deformation control unit;
a head end of the needle body sleeve is connected to an outer edge of the head end of the suture line sleeve in a fixed sealing manner, parts of the deformation control units extending into the wound are isolated from organ tissues in the body of a surgical subject by the needle body sleeve, and a line feeding unit extending into the wound is also isolated from organ tissues in the body of a surgical subject by the needle body sleeve;
a tail end of the short needle is connected to the suture line;
the heat supply unit is connected to the tail end of the conductive metal wire and used to provide a heat source;
the power supply unit is connected to the tail end of the conductive metal wire and the head end of the electrical loop metal wire and used to provide a power source;
the tail end of the memory metal wire is electrically connected to the head end of the conductive metal wire to form the deformation control unit core body of the single deformation control unit, a head end of the deformation control unit core body is the head end of the memory metal wire, and a tail end of the deformation control unit core body is the tail end of the conductive metal wire;
the tail end of the electrical loop metal wire is electrically connected to the head end of the memory metal wire, and located outside the film sleeve;
the film sleeve has a length greater than a length of the deformation control unit core body wrapped inside the film sleeve;
an inner diameter of the film sleeve is greater than an outer diameter of the deformation control unit core body;
the film sleeve is made of an insulating, flame-retardant material;
an elastic module of the memory metal wire is not lower than that of the conductive metal wire;
the memory metal wire is made of a memory metal that is elastically deformed as a temperature changes or a current changes, and a radian of the memory metal wire is elastically deformed as the temperature changes or the current changes;
the aerogel particles are made of a low-density solid heat-insulating material;
the head end of the needle body sleeve extends into the minimally invasive surgical wound during surgery, and the length of the needle body sleeve satisfies that the tail end of the needle body sleeve is always located outside the minimally invasive surgical wound during surgery;
the electrical loop metal wire is located inside the needle body sleeve and outside the suture line sleeve; and
the lengths of the memory metal wire, the conductive metal wire and the electrical loop metal wire satisfy that the tail end of the conductive metal wire and the head end of the electrical loop metal wire are located outside the minimally invasive surgical wound during surgery.

2. The modular deformable suture needle for a minimally invasive surgery according to claim 1, characterized in that a friction coefficient and an elastic modulus of the film sleeve satisfy that the strain of the film sleeve in a length direction is lower than 5% of the strain of the deformation control unit core body in a length direction when the deformation control unit core body in the film sleeve is bent, stretched or contracted.

3. The modular deformable suture needle for a minimally invasive surgery according to claim 1, characterized in that the radians of the conductive metal wire and the electrical loop metal wire do not change as the temperature changes or the current changes, and side surfaces of the conductive metal wire and the electrical loop metal wire are coated with an insulating coating.

4. The modular deformable suture needle for a minimally invasive surgery according to claim 1, characterized in that a largest Feret diameter of the aerogel particles is less than 0.1 mm.

5. The modular deformable suture needle for a minimally invasive surgery according to claim 1, characterized in that the arrangement of the control units and the line feeding unit in the flexible deformation needle body composed of parallel units with position differences also satisfies a minimum section diameter at the section of the maximum diameter of the flexible deformation needle body composed of parallel units with position differences.

6. The modular deformable suture needle for a minimally invasive surgery according to claim 5, characterized in that:
when I=1 and the quantity of the line feeding unit is 1, the deformation control unit is adjacent to the line feeding unit at the cross section of the needle body;
when I=2 and the quantity of the line feeding unit is 1, the two deformation control units are adjacent to each other at the cross section of the needle body and both adjacent to the line feeding unit; and
when I≥3 and the quantity of the line feeding unit is 1, the deformation control units are located outside the line feeding unit at the cross section of the needle body.

7. The modular deformable suture needle for a minimally invasive surgery according to claim 1, characterized in that the control units and the line feeding unit in the flexible deformation needle body composed of parallel units with position differences also satisfy:
$$\vert l - L , ( 1 ) \vert \times 10 \leq L , \qquad ( 1 )$$
wherein l represents a length of the suture line sleeve extending into the minimally invasive surgical wound.

8. The modular deformable suture needle for a minimally invasive surgery according to claim 1, characterized in that the form control method of the suture needle comprises the following steps:
obtaining a quantitative relationship between bending deformation features of each deformation control unit and a heat power provided by the corresponding heat supply unit or a voltage provided by the corresponding power supply unit through a statistical learning model;
implementing turning control on the corresponding memory metal wire by rotating the tail end of the deformation control unit and adjusting the corresponding heat power or voltage;
implementing coherent form control to move forward by controlling the voltages or heat powers of the deformation control units in an order of i increment or by rotating the tail end of the deformation control unit; and
implementing coherent form control to move backward by controlling the voltages or heat powers of the deformation control units in an order of i decrement or by rotating the tail end of the deformation control unit.

9. The modular deformable suture needle for a minimally invasive surgery according to claim 8, characterized in that the statistical learning model is a nonlinear function.

10. The modular deformable suture needle for a minimally invasive surgery according to claim 8, characterized in that:
the bending deformation features of the i^{th} deformation control unit comprise an arc-chord ratio αi, a peak deviation position βi, and are mouth directions, the are mouth directions comprising a first direction φi′δiζi and a second direction θi′δiζi, wherein
a central axis of the i^{th} deformation control unit is divided into a deformable segment and a non-deformable segment according to a boundary between the memory metal wire and the conductive metal wire of the i^{th} deformation control unit;
a space rectangular coordinate system oi-xiyizi is defined: a normal plane of the non-deformable segment on the central axis of the i^{th} deformation control unit at the boundary between the deformable segment and the non-deformable segment is taken as a coordinate plane zi-oi-yi, the central axis of the i^{th} deformation control unit passes through an origin oi, and a normal vector of the coordinate plane zi-oi-yi passing through the origin oi is taken as an xi axis;
a spherical coordinate system is defined as oi-riφθii, and a conversion relationship between the two coordinate systems is as follows: coordinates of a point A in the rectangular coordinate system are set as (xi, yi, zi), spherical coordinates of the point A are set as (ri, φi, θi), the coordinate ri is a distance from the point A to the origin oi, and φi is an included angle formed between a half-plane passing through zi axis and point A and the coordinate plane zi-oi-xi; θi is an included angle between a line segment oiA and a positive direction of the zi axis;
the origin oi is a tail end point of the deformable segment on the central axis of the i^{th} deformation control unit; a farthest point on the central axis of the i^{th} deformation control unit from the origin oi is defined as a head end point gi of the deformable segment on the central axis of the i^{th} deformation control unit; a distance between gi and oi along the central axis of the i^{th} deformation control unit is a length of the deformable segment on the central axis of the i^{th} deformation control unit; a midpoint of the deformable segment on the central axis of the i^{th} deformation control unit is denoted as mi;
$$α i = \frac{L g ioi}{l g ioi}$$
wherein Lgioi represents the length of the deformable segment on the central axis of the i^{th} deformation control unit;
lgioi is a length of a straight segment gioi;
$$β i = \frac{2 η i \times γ i}{Lgioi}$$
$$η i = \{ \begin{matrix}1 , lmioi > lmigi \\ - 1 , lmioi < lmigi\end{matrix} .$$
wherein γi is the shortest distance from mi to an intermediate surface;
the intermediate surface is a normal plane of the straight segment gioi passing through ζi;
ζi is a midpoint of the straight segment gioi;
ηi is a parameter of deflection;
lmioi is a length of a straight segment mioi;
lmigi is a length of a straight segment migi;
$$φ i {}^{′} δ i ζ i = φ i {}^{′} δ i ζ {i}^{′} v / 1 rad$$
$$θ i {}^{′} δ i ζ i = θ i {}^{′} δ i ζ {i}^{′} v / 1 rad$$
wherein spherical coordinates of a vector δiζi are (ri′δiζi′v, φi′δiζi′v, θi′δiζi′v);
δi is an arc bottom point, specifically an intersection point of the intermediate surface and the deformable segment on the central axis of the i^{th} deformation control unit; and
in the minimally invasive surgery, on the premise that a part of the modular deformable suture needle for the minimally invasive surgery extending into the wound is able to smoothly turn, move forward, move backward or make other actions, the workload in the coherent form control is minimum, that is, the lowest overall bending deformation degree Σαi of the part of the modular deformable suture needle for the minimally invasive surgery extending into the wound is taken as a control object, and Σαi is a sum of αi of the deformation control units where all memory metal wires extending into the minimally invasive surgical wound are located.
